(19) United States
(12) Patent Application Publication
(10) Pub. No.: US 2025/0177957 A1
(43) Pub. Date: Jun. 5, 2025

(54) PROCESS FOR THE TRANSFORMATION OF A VANADIUM/PHOSPHORUS MIXED OXIDE CATALYST PRECURSOR INTO THE ACTIVE CATALYST FOR THE PRODUCTION OF MALEIC ANHYDRIDE

(71) Applicant: POLYNT S.P.A., SCANZOROSCIATE (IT)

(72) Inventors: Carlotta CORTELLI, ZOLA PREDOSA (IT); Laura FRATALOCCHI, MILANO (IT); Silvia LUCIANI, COLLI DEL TRONTO (IT); Laura SETTI, SAN CESARIO SUL PANARO (IT); Fabrizio CAVANI, MODENA (IT); Lorenzo GRAZIA, BERGAMO (IT); Tommaso TABANELLI, BOLOGNA (IT)

(73) Assignees: POLYNT S.P.A., SCANZOROSCIATE (IT); ALMA MATER STUDIORUM - UNIVERSITA' DI BOLOGNA, BOLOGNA (IT)

(21) Appl. No.: 18/842,965

(22) PCT Filed: Dec. 29, 2022
(86) PCT No.: PCT/EP2022/088041

§ 371 Date: Aug. 30, 2024

(30) Foreign Application Priority Data
Mar. 2, 2022 (IT) 102022000003872

(51) Int. Cl. B01J 23/22; B01J 37/08; B01J 37/10; B01J 37/12; C07C 51/215

(52) U.S. Cl. CPC B01J 23/22; B01J 37/082; B01J 37/10; B01J 37/12; C07C 51/215; C07C 2523/22

(57) ABSTRACT

A process for the activation of a vanadium/phosphorus mixed oxide catalyst (“VPO” catalyst) precursor, whereby the precursor is transformed into the corresponding active catalyst suitable for use in the production of maleic anhydride by partial oxidation of n-butane.

The present invention relates to a process for the transformation of a vanadium/phosphorus mixed oxide catalyst precursor into the active catalyst for the production of maleic anhydride. The invention further relates to an active catalyst obtainable with the above mentioned transformation process, and to a process for manufacturing maleic anhydride using such active catalyst.

Maleic anhydride is a well-known and versatile intermediate used in the production of a large number of different chemical products, including unsaturated polyester resins, pharmaceutical products and agrochemical products.

Initially, industrial-scale production of maleic anhydride was carried out by the selective oxidation of benzene with catalysts based on oxides of vanadium/molybdenum. Nowadays, benzene has for the most part been replaced by non-aromatic hydrocarbons, in particular n-butane, as a starting raw material.

The process of selective oxidation of n-butane to maleic anhydride is conducted in the gaseous phase, in the presence of a vanadium and phosphorus mixed oxide catalyst (so-called “VPO” catalyst) which comprises vanadyl pyrophosphate of formula (VO)₂P₂O₇ as main component, using both fluidized bed reactor technology and fixed bed reactor technology. On an industrial scale, the process is typically conducted at a conversion of n-butane in a range of 80-86%, with yields in weight of maleic anhydride of 96-103%. The main byproducts of the process are CO and CO₂ (CO_{X}), but acetic acid and acrylic acid are also formed with yields in weight of 2.5-3%.

Production of the VPO catalyst is divided into two main steps: (i) the synthesis of the precursor vanadyl acid orthophosphate hemihydrate of formula (VO)HPO₄·0.5H₂O through the reduction of a pentavalent form of vanadium (commonly vanadium pentoxide) in the presence of a phosphorus source (commonly phosphoric acid), and (ii) transformation of the precursor to vanadyl pyrophosphate (“VPP”) by means of a thermal treatment. This second step is also called the “activation process” and it is known to have a strong influence on the catalytic performance of the resulting active catalyst.

During the activation, a topotactic transformation of the precursor takes place, with cis-trans rearrangement of the vanadyl dimers to form the active phase (i.e. VPP) while retaining the morphology. In this transformation, the V—O and P—O bonds remain unaltered, while the weaker V-H₂O and P—H₂O bonds are broken, thus enabling the condensation of orthophosphate groups to pyrophosphate.

During the activation, oxidation takes place also of the organic residues present on the precursor (residues of the organic solvents used in the synthesis of the precursor and not burned during the precalcination) to CO_{X}, as well as the oxidation of part of the VPP, in which vanadium has an oxidation number of +4 (V⁴⁺), to form VOPO₄ phases in which vanadium has an oxidation number of +5 (V⁵⁺).

The co-presence of VPP and of the VOPO₄ phases in the active phase of the catalyst is known in the literature, and appears to be due to the fact that the crystalline VPP usually remains on the edges of the active phase, while the amorphous VPP and the VOPO₄ phases remain at the core. Among the latter, the formation of δ-VOPO₄ is particularly desired, as it is the most selective for maleic anhydride. It has been observed that δ-VOPO₄ can subsequently be reconverted to VPP or to the αI- and αII-VOPO₄ phases; these phases promote the activity only if they are present in traces, but are not selective for maleic anhydride [Cavani et al., Chem. A Eur. J., 16(5), 1646-1655 (2010)].

The various transformations may not be complete, thus leaving traces of different VOPO₄ phases in the catalyst. The above mentioned αI-, αII- and δ-VOPO₄ phases are not the only possible V⁵⁺ species present in the catalyst. In fact, the presence is known also of the β-VOPO₄ phase, which is inactive in the reaction to synthesize maleic anhydride, and of the ω—VOPO₄ phase, the conversion of which to δ-VOPO₄ is often favored under reaction conditions.

The thermal treatment of the activation process comprises two steps. In the first step, the water of crystallization is lost and the crystalline structure of vanadyl acid phosphate is retained, while in the second step the orthophosphate functional groups are condensed to form the characteristic groups of pyrophosphate with a substantial liberation of water [Cavani et al., Catal. Today, 32(1-4), 125-132(1996)].

Usually the above mentioned thermal treatment is subdivided in turn into two steps. The first step entails drying at temperatures below 300° C. in order to eliminate the volatile residues deriving from the synthesis, but without eliminating the water of crystallization. The second step consists of the actual step of dehydration and calcination. The prior art describes different examples of thermal treatments: (i) dehydration in situ inside the reactor, feeding the air-butane mixture at a low contact time and subsequent increase of temperature to the standard reaction conditions, (ii) dehydration in the absence of oxygen at high temperature (>400° C.) and subsequent treatment in an air-butane mixture [Johnson et al., J. Ma. Chem. Soc., 106(26), 8123-8128 (1984)], (iii) calcination at high temperature (>400° C.) and subsequent treatment in the reaction mixture, or (iv) hydrothermal treatment entailing a first step up to 280° C., feeding an air/steam mixture and a second step up to 390° C., feeding nitrogen [Cornaglia et al., Appl. Catal., 74(1), 15-27 (1991)].

According to the literature, many parameters associated with the activation phase can directly influence the properties of the vanadyl pyrophosphate obtained, such as the temperature ramp of the treatment, the duration of the treatment, the composition of the feed stream, the crystallinity, the morphology and the composition (i.e. the phosphorus/vanadium atomic ratio, the presence of doping elements, and the carbon content) of the initial precursor [Horowitz, H. S.; Blackstone, C. M.; Sleight, A. W.; Teufer, G., Appl. Catal., 38(2), 193-210 (1988)].

Even though the prior art has already made available the above mentioned processes for activating the precursor, the need still remains to provide improved methods for transforming the VPO catalyst precursor into the active catalyst, which advantageously make it possible to obtain VPO catalysts with improved catalytic performance, in particular in the synthesis of maleic anhydride by partial oxidation of n-butane.

In light of the foregoing, the aim of the present invention is to provide an improved process for the transformation of a VPO catalyst precursor into the active catalyst for the production of maleic anhydride.

Within this aim, an object of the invention is to provide a process for transforming the precursor into an active VPO catalyst that is capable of imparting to the catalyst improved catalytic performance with respect to the performance obtained with the activation processes of the prior art.

Another object of the invention is to provide a VPO catalyst with improved catalytic performance, so as to obtain a yield of maleic anhydride that exceeds the yield of the current generation of VPO catalysts.

Yet another object of the invention is to provide an improved process for producing maleic anhydride with high yield.

This aim and these and other objects which will become more apparent hereinafter are achieved by:
a process for the transformation of a vanadium and phosphorus mixed oxide catalyst precursor into an active catalyst for the synthesis of maleic anhydride, according to claim 1,
an active catalyst according to claim 9, obtainable by the above mentioned transformation process, and
a process according to claim 10 for the production of maleic anhydride by partial oxidation of n-butane in an oxygen-containing gas mixture in the presence of the above mentioned active catalyst.

Further characteristics and advantages of the invention will become better apparent from the detailed description that follows.

In a first aspect, the present invention relates to an improved process for the transformation of a VPO catalyst precursor into an active catalyst, by means of which the catalytic performance of the active catalyst is increased with respect to catalysts activated using the known processes.

The inventors of the present invention have in fact investigated the effect of varying the operative parameters of the process of transformation of the precursor into the active catalyst (such as the composition of the feed stream, the temperature ramp, and the contact time between reagent gas and catalyst) and, without wishing to be bound to any particular theory, they have concluded that varying one or more of these parameters appears to have an effect on the type and abundance of the V⁵⁺ and V⁴⁺ species present in the active phase of the catalytic material, thereby influencing the performance of the activated catalyst.

According to the present invention, the transformation process comprises a step of a) initial heating of the precursor to an initial temperature of 170-190° C. and holding at the initial temperature for 1-10 minutes, in an atmosphere of air, i.e. an atmosphere composed of 100% by volume of air.

Preferably, in this step the initial temperature is 180° C. Preferably, the initial temperature is held for 2 minutes. More preferably, the precursor is heated to the initial temperature of 180° C. and held at that initial temperature for 2 minutes.

Preferably, the initial heating is carried out at a heating rate of from 0.1° C./minute to 10° C./minute, more preferably from 1° C./minute to 4° C./minute, even more preferably 2° C./minute.

After the step a) of initial heating and holding at the initial temperature, the process of the invention may comprise an optional step b′) of heating the precursor from the initial temperature to a calcination temperature TC1 of 320-380° C. and holding at the temperature TC1 for 0.5-3 hours, in an atmosphere selected from an atmosphere composed of 10%-50% by volume of air and 50%-90% by volume of steam, and an atmosphere composed of 20%-40% by volume of air, 5%-15% by volume of steam and 45%-75% by volume of an inert gas.

In a preferred embodiment of the process according to the invention, the optional step b′) is carried out. In a more preferred embodiment, the step b′) is carried out in an atmosphere composed of 10%-50% by volume of air and 50%-90% by volume of steam, even more preferably composed of 30% by volume of air and 70% by volume of steam. In another more preferred embodiment, the step b′) is carried out in an atmosphere composed of 20%-40% by volume of air, 5%-15% by volume of steam and 45%-75% by volume of an inert gas, even more preferably 30% by volume of air, 10% by volume of steam and 60% by volume of an inert gas.

Preferably, in step b′) the calcination temperature TC1 is 350° C. Preferably, the temperature TC1 is held for 1-2 hours, more preferably for 1 hour. More preferably, in step b′) the precursor is heated to the calcination temperature TC1 of 350° C. and is held at the temperature TC1 for 1 hour.

Preferably, in step b′) the heating is carried out at a heating rate of from 0.1° C./minute to 10° C./minute, more preferably from 1° C./minute to 4° C./minute, even more preferably 2° C./minute.

After step a) or, if the optional step b′) is carried out, after step b′), the process of the invention comprises a step b) of further heating of the precursor from the initial temperature (when step b′) is not carried out) or from the calcination temperature TC1 (when step b′) is carried out) to a calcination temperature TC2 of 390-460° C. and holding at the calcination temperature TC2 for 0.5-3 hours, in an atmosphere the composition of which depends on whether step b′) has been carried out or not.

In particular, when step b′) is carried out the atmosphere in step b) has the same composition as the atmosphere in step b′). Therefore, in embodiments of the process wherein step b′) is carried out, in a first alternative steps b′) and b) both use an atmosphere composed of 10%-50% by volume of air and 50%-90% by volume of steam, preferably 30% of air and 70% of steam, and in a second alternative steps b′) and b) both use an atmosphere composed of 20%-40% by volume of air, 5%-15% by volume of steam and 45%-75% by volume of an inert gas, preferably 30% of air, 10% of steam and 60% of an inert gas.

If on the other hand step b′) is not carried out, then the atmosphere in step b) is composed of 20%-40% by volume of air, 5%-15% by volume of steam and 45%-75% by volume of an inert gas, preferably 30% of air, 10% of steam and 60% of an inert gas.

Preferably, in step b) the calcination temperature TC2 is 425° C. Preferably, the calcination temperature TC2 is held for 1-2 hours, more preferably 2 hours. More preferably, in step b′) the precursor is heated to the calcination temperature TC2 of 425° C. and is held at that temperature for 2 hours. Such preferred temperatures and times for step b) apply irrespective of whether or not the process of the invention comprises the optional step b′).

Preferably, in step b) the heating to the calcination temperature TC2 is carried out at a heating rate of from 0.1° C./minute to 10° C./minute, more preferably from 1° C./minute to 4° C./minute, even more preferably 2° C./minute.

After step b) of heating the precursor to the calcination temperature TC2 and holding at that temperature, the process of the invention comprises a step c) of heating the precursor from the calcination temperature TC2 to a calcination temperature TC3 of 470-550° C. and holding at the calcination temperature TC3 for 0.5-3 hours, in an atmosphere composed of 100% by volume of an inert gas.

Preferably, the calcination temperature TC3 is 500° C. Preferably, the calcination temperature TC3 is held for 1-2 hours, more preferably 2 hours. More preferably, in step c) the precursor is heated to the calcination temperature TC3 of 500° C. and is held at that calcination temperature TC3 for 2 hours.

Preferably, in step c) the heating to the calcination temperature TC3 is carried out at a heating rate of from 0.1° C./minute to 10° C./minute, more preferably from 1° C./minute to 4° C./minute, even more preferably 2° C./minute.

At the end of step c) of heating the precursor to the calcination temperature TC3 and holding at that temperature, the active VPO catalyst is obtained.

Once the active VPO catalyst is obtained, the process according to the invention entails a step d) of cooling, in which the active catalyst is brought to ambient temperature. Although the conditions of this step are not critical, it is preferably carried out in an atmosphere composed of 100% by volume of an inert gas and the cooling rate preferably does not exceed 5° C./minute.

In a particularly preferred embodiment where step b′) is carried out, the process according to the invention comprises the steps of:
a) initial heating of the precursor to an initial temperature of 180° C. and holding at the initial temperature for 2 minutes, in an atmosphere composed of 100% by volume of air;
b′) heating the precursor from the initial temperature to a calcination temperature TC1 of 350° C. and holding the calcination temperature TC1 for 1 hour in an atmosphere composed of 30% by volume of air and 70% by volume of steam;
b) heating the precursor from the calcination temperature TC1 to a calcination temperature TC2 of 425° C. and holding at the calcination temperature TC2 for 2 hours in an atmosphere composed of 30% by volume of air and 70% by volume of steam;
c) heating of the precursor from the calcination temperature TC2 to a calcination temperature TC3 of 500° C. and holding at the calcination temperature TC3 for 2 hours, in an atmosphere composed of 100% by volume of an inert gas; and
d) cooling the active catalyst obtained in step c).

In another, particularly preferred embodiment where instead step b′) is not carried out, the process according to the invention comprises the steps of:
a) initial heating of the precursor to an initial temperature of 180° C. and holding at the initial temperature for 2 minutes, in an atmosphere composed of 100% by volume of air;
b) heating the precursor from the initial temperature to a calcination temperature TC2 of 425° C. and holding at the calcination temperature TC2 for 2 hours, in an atmosphere composed of 30% by volume of air, 10% by volume of steam and 60% by volume of an inert gas;
c) heating of the precursor from the calcination temperature TC2 to a calcination temperature TC3 of 500° C. and holding at the calcination temperature TC3 for 2 hours, in an atmosphere composed of 100% by volume of an inert gas; and
d) cooling the active catalyst obtained in step c).

In general, in the steps of the process of the invention in which an inert gas is used, the inert gas may be selected from nitrogen and a noble gas (for example helium or argon), however, preferably the inert gas is nitrogen.

In the steps of the process of the invention in which an atmosphere comprising steam is used, the content by volume of steam is calculated as H₂O.

Furthermore, in the steps of the process of the invention in which an atmosphere comprising or consisting of air is used, it is possible to use molecular oxygen as an alternative to air, without for this reason departing from the claimed scope of protection, on condition that the percentage by volume of oxygen in the resulting atmosphere corresponds to the percentage that is obtained when the quantity of air indicated for a given step of the process is used, substituting the remaining percentage by volume with an inert gas (for example nitrogen). Therefore, where a step of the process of the invention uses an atmosphere composed of 100% by volume of air, alternatively it is possible to use an atmosphere composed of 21% by volume of molecular oxygen and of an inert gas for the remaining part, up to 100% by volume. Where a step of the process of the invention instead uses an atmosphere composed, for example, of 30% by volume of air, 10% by volume of steam and 60% by volume of an inert gas, alternatively it is possible to use an atmosphere composed of 6.3% by volume of molecular oxygen, 10% by volume of steam and an inert gas for the remaining part, up to 100% by volume.

With regard to the preparation of the precursor comprising vanadyl acid orthophosphate hemihydrate as the main component to be activated using the transformation process of the invention, substantially all the preparation methods described in the background art can be used.

In particular, the known methods for preparing the precursor of the catalyst (see for example U.S. Pat. No. 5,137,860 and EP 804963 A1) conventionally require the reduction of a pentavalent vanadium source (for example V₂O₅ or suitable precursors such as for example ammonium metavanadate, vanadium chloride, vanadium oxychloride, vanadyl acetylacetonate, vanadium alkoxides) in conditions that lead the vanadium to a tetravalent state (average oxidation number +4), and the reaction of the tetravalent vanadium with a phosphorus source (for example H₃PO₄).

As a reducing agent, it is possible to use organic or inorganic compounds. In the “VPA” method, hydrochloric acid is used as a reducing agent, instead in the “VPO” method organic alcohols are used, while in the “VPD” method water is used, with the initial formation of vanadyl phosphate dihydrate (VOPO₄·2H₂O) which is then reduced to the precursor. Isobutyl alcohol is the most frequently used reducing agent, optionally mixed with benzyl alcohol.

In the preparation of VPO catalysts promoted with one or more promoter elements, each promoter element can be added in the form of a suitable precursor, for example of the acetylacetonate type or other commercially-known and used compounds or salts of the promoter element.

By way of example, the precursor of the VPO catalysts can be prepared according to the method described in PCT publication WO 00/72963. In accordance with this method, the vanadium source and the phosphorus source react in the presence of an organic reducing agent which comprises (a) isobutyl alcohol, optionally mixed with benzyl alcohol, and (b) a polyol, in a weight ratio (a):(b) comprised between 99:1 and 5:95.

The precursor is then filtered, washed and optionally dried, preferably at a temperature between 120° C. and 200° C.

After its preparation as above, the precursor may be subjected to pelletization, granulation and tableting.

Furthermore, the precursor can be subjected to precalcination prior to its transformation into the active catalyst according to the transformation process of the invention. Therefore, in an embodiment of the invention the precursor is precalcined before the step a) of initial heating, preferably to a precalcination temperature of 200-300° C. in an atmosphere of air.

A second aspect of the present invention is an active VPO catalyst for the synthesis of maleic anhydride obtainable by any of the embodiments of the transformation process described herein.

The active VPO catalyst of the present invention comprises vanadyl pyrophosphate of formula (VO)₂P₂O₇ as the main component and, compared to VPO catalysts activated with transformation processes of the prior art, is characterized by improved catalytic performance, by virtue of which a higher yield of maleic anhydride is obtained. Without wishing to be bound to any particular theory, the inventors of the present invention believe that this improvement is a consequence of the particular type and abundance of V⁵⁺ and V⁴⁺ phases in the active phase, which are obtained by virtue of the specific values of the operative parameters (temperature ramp, partial pressure of oxygen and of steam in the feed stream, and oxygen/steam molar ratio) used in the transformation process of the invention.

In a preferred embodiment, the active catalyst of the present invention comprises at least one promoter element. Each promoter element can be selected from the group consisting of the elements of groups 1, 2, 3, 4, 5, 6, 7, 11, 12, 13, 14, 15 and 16 of the periodic table of the elements. The term “periodic table of the elements” refers to the periodic table of the elements in the version dated 1 Dec. 2018 published by IUPAC-International Union of Pure and Applied Chemistry (accessible at the following URL: https://iupac.org/what-we-do/periodic-table-of-elements/periodic-table-archives/).

Preferably, the promoter element is selected from the group consisting of lithium, titanium, zirconium, niobium, molybdenum, tungsten, iron, cobalt, copper, bismuth, and mixtures thereof. More preferably, the promoter element is selected from the group consisting of niobium, molybdenum, iron, cobalt, copper, bismuth, and mixtures thereof.

In a preferred embodiment, the active catalyst of the present invention comprises a first promoter element selected from the group consisting of cobalt, iron, copper, and mixtures thereof, optionally a second promoter element selected from either bismuth or niobium, and optionally molybdenum as a third promoter element.

When the second promoter element is niobium, the catalyst is preferably used for the conversion of n-butane to maleic anhydride in a fluidized bed reactor, and when the second promoter is bismuth, the catalyst is preferably used for the conversion of n-butane to maleic anhydride in a fixed bed reactor.

Advantageously, the presence of molybdenum as a third promoter element makes it possible to decrease the yield of acrylic acid, limiting the content of this undesired byproduct to amounts lower than 1% by weight and without compromising the yield of maleic anhydride.

Once activated using the transformation process of the present invention, the catalyst is ready to be used in a process for the production of maleic anhydride according to a third aspect of the invention.

According to such third aspect of the invention, the production of maleic anhydride is carried out by partial oxidation of n-butane in a mixture with an oxygen-containing gas (for example air or molecular oxygen) in the presence of the VPO catalyst obtainable by the transformation process of the present invention.

The reactor used for the production of maleic anhydride can be of the fixed bed or fluidized bed type, as a function of the geometry of the VPO catalyst.

The initial concentration of n-butane in the mixture with the oxygen-containing gas (i.e. the concentration of n-butane in the reactor feed) is generally comprised in a range from 1.00 to 4.30 mol %. The initial concentration of n-butane can be comprised between 1.00 and 2.40 mol %, preferably between 1.65 and 2.20 mol %, for example when the process is performed in a fixed bed reactor. Alternatively, the initial concentration of n-butane can be comprised between 2.50 and 4.30 mol %, for example when the process is performed in a fluidized bed reactor.

Preferably, the oxidation reaction is performed at a temperature from 320° C. to 500° C., more preferably from 400° C. to 450° C.

The invention will now be described with reference to the following non-limiting examples.

### EXAMPLES

In order to evaluate the effect of varying the operative parameters of the activation process on the chemical-physical properties and on the catalytic performance of the resulting catalyst, two batches of vanadyl acid orthophosphate hemihydrate precursor were prepared using the procedure described below. From these two batches of precursor 14 active VPO catalysts were then obtained, which differ in the operative conditions adopted for their activation (Table 2).

### 1) Synthesis of the Catalyst Precursors

The synthesis of each of the two batches of precursor was carried out in a 30 L reaction flask, fitted with a heating jacket and reflux condenser, in which was placed 19.90 L of isobutyl alcohol, followed by the addition of 1.945 kg of vanadium pentoxide and 2.458 kg of phosphoric acid 100%.

The reaction was carried out at a temperature of approximately 106-110° C., keeping the system in total reflux for approximately 8 hours. At the end of the reaction, a product was obtained with the bright blue color of vanadyl acid orthophosphate hemihydrate. This product was removed from the flask and filtered through a Büchner funnel for approximately 6 hours. The solid residue (cake) resulting from filtration was placed in a tray and dried at ambient temperature for 24 hours. The material was then subjected to further drying at 150° C. for 8 hours and then precalcined at 220° C. for 3 hours and at 260° C. for 3 hours in an oven in static air.

The precalcined precursor was then subjected to manual grinding in a mortar before being subjected to the activation process.

Table 1 below summarizes the main characteristics of the two batches of precursor that were prepared. These characteristics are representative of the extremes of the natural range of variability that characterizes the production process of the precursor on an industrial scale.

TABLE 1

| | Phosphor/vanadium | Carbon content | V⁵⁺ content |
|---|---|---|---|
| Precursor | atomic ratio | [ppm] | [%] |
| A | 1.15 | 9490 | 0.006 |
| B | 1.20 | 6344 | 0.040 |

### 2) Setup of the Activation Tests

The activation of the precursors was carried out on a laboratory scale, in a continuous fixed bed rig with the possibility to vary the composition of the feed stream. This rig consists of a micro-reactor with an inner diameter (ID) of 1.4 cm inserted into an electric resistance oven. For each activation, an amount of 2.0 g of precursor was loaded in the micro-reactor, for a height of the catalytic bed of 1.60 cm, which corresponds to a contact time of 5 seconds. A thermocouple was placed inside the catalytic bed, at the center (depth≈0.80 cm), and was used to regulate the temperature ramp set for the oven.

The precursors A and B were transformed into a total of 14 active catalysts using an activation process that can be generally outlined as follows:
Step 1, heating from ambient temperature to 180° C. at a heating rate of 2° C./min and holding the temperature at 180° C. for 2 minutes, with a feed stream composed of 100% by volume of air;
Step 2, heating from 180° C. to 425° C. at a heating rate of 2° C./min and holding the temperature at 425° C. for 2 hours, with a feed stream having the composition shown in Table 2 for each one of the 14 activation tests carried out;
Step 3, heating from 425° C. to 500° C. at a heating rate of 2° C./min and holding the temperature at 500° C. for 2 hours, with a feed stream composed of 100% by volume of nitrogen.

The activation tests carried out differ from each other in the composition of the feed stream or in the temperature ramp of step 2, while steps 1 and 3 remained unaltered for all tests. This choice derives from previous studies carried out by the inventors, following which it was observed that the crucial step of the transformation of the precursor to vanadyl pyrophosphate occurs in the temperature range of step 2.

TABLE 2

Step 2 feed stream [vol %]

| Cat. | Precursor | Steam | Air (O₂) | N₂ |
|---|---|---|---|---|
| 1 | A | — | 100 (21.0) | — |
| 2 | A | 10 | 90 (18.9) | — |
| 3 | A | 40 | 60 (12.6) | — |
| 4 | A | 70 | 30 (6.3) | — |
| 5 | A | — | 60 (12.6) | 40 |
| 6 | A | — | 30 (6.3) | 70 |
| 7* | A | 10 | 30 (6.3) | 60 |
| 8 | A | 40 | 30 (6.3) | 30 |
| 9* | A | 70 | 30 (6.3) | — |
| 10 | B | — | 100 (21.0) | — |
| 11 | B | 10 | 90 (18.9) | — |
| 12 | B | 40 | 60 (12.6) | — |
| 13 | B | 70 | 30 (6.3) | — |
| 14* | B | 10 | 30 (6.3) | 60 |

In Table 2, the catalysts marked with “*” were activated using a process according to the invention, and the remaining catalysts are comparatives.

### 3) Chemical-Physical Characterization of the Activated Catalysts

The catalysts activated as described above were subjected to chemical-physical characterization for the purpose of studying the effect deriving from the modification of the following parameters of the activation process: i) relative air: steam composition; ii) partial pressure of oxygen; iii) partial pressure of steam; iv) transformation temperature of the precursor.

In particular, for the characterization the following analyses were carried out:
X-ray diffraction (XRD) to study the bulk crystalline phases, using a Philips PW 1050/81 diffractometer (source Cu Kα, λ=1.5406 Å);
Raman spectroscopy to study the surface crystalline phases, using a Renishaw Raman System RM1000, fitted with a Leica DLML microscope, with magnifications of 5×, 20× and 50×, video camera, CCD detector and argon ion laser source (514 nm) with 25 mW power;
BET analysis to measure the surface area (SSA), using a Micromeritics ASAP 2420 device;
X-ray fluorescence (XRF) to determine the elementary chemical composition (and specifically the P/V ratio), using a Bruker AXS S4 Explorer spectrometer;
determination of the average valency of vanadium (Vox), using redox titration after dissolving the catalyst.

Since catalyst 4 was activated using the activation procedure that is currently in use, this catalyst was used as the “reference sample”. From the point of view of the chemical-physical characterization carried out on the fresh material, catalyst 4 shows an SSA of 13 m²/g and a Vox of 4.21. Analysis of the bulk crystalline phase using XRD revealed a crystalline material, its main phase consisting of VPP and ω—VOPO₄, while the Raman analysis of the surface crystalline phase showed a main phase consisting of VPP with traces of VOPO₄·2H₂O and of ω—VOPO₄.

Effects of varying the relative air: steam composition—Catalysts from 1 to 4 were prepared to evaluate the effect of varying, in terms of relative proportions, the content of steam and air (and therefore oxygen) in the feed stream.

The characterization of the obtained fresh catalysts shows that as the steam content in the mixture is increased, or as the percentage of oxygen is decreased, the surface area increases and then stabilizes, while there is a marked effect on the average valency of vanadium. Catalysts 1 and 2, which were activated respectively in the absence of steam and with 10% steam, are in fact characterized by a high V⁵⁺ content, given the valencies of 4.52 and 4.53. By contrast, in catalysts 3 and 4 it was observed that as the steam increases (i.e. as the oxygen decreases), the content of vanadium in the oxidation state 5+ decreases to the average valency value of 4.21, which was also observed for the reference sample.

Turning to the crystalline phases, the trend reflects that of the valency of vanadium, catalysts 1 and 2 are in fact characterized by the presence, both in bulk and on the surface, of V⁵⁺ phases (mainly ω—VOPO₄ and VOPO₄·2H₂O with traces of αI-VOPO₄) and VPP only in traces. In catalysts 3 and 4, the presence of V⁵⁺ phases decreases progressively as VPP increases. In conclusion, as the oxygen content decreases for an increase in the steam content, a decrease is thus observed in the formation of V⁵⁺ phases in favor of an increase of the active VPP phase.

Effect of the partial pressure of oxygen—Catalysts 5-6, compared with catalyst 1, make it possible to individually assess the effect of the partial pressure of oxygen.

Thus, it was observed that as the partial pressure of oxygen decreases, there is a progressive decrease of the average valency of vanadium: from 4.52 for catalyst 1 activated in the presence of air alone, there is a drop to 4.25 for catalyst 6 activated with a 30:70 mixture of air: nitrogen. In this case too, the crystalline phases of the catalysts are in agreement with the trend of the V⁵⁺ content. In fact, catalysts 1 and 5 (with higher valency) consist mainly of ω—VOPO₄ and there are only traces of αI-VOPO₄ and VPP, while catalyst 6 comprises VPP as the main phase and, in addition, ω—VOPO₄ and δ-VOPO₄.

Catalysts 1, 5 and 6 are furthermore all characterized by low crystallinity, independently of the partial pressure of oxygen. The inventors believe that this characteristic is due to the absence in the feed stream of steam, which it is thought may be a promoter of crystallinity.

Effect of the partial pressure of steam—Catalysts 7 and 8, compared with catalysts 4 and 6, make it possible to assess the effect of the partial pressure of steam (for the same oxygen content and using nitrogen for the remaining part).

Thus, it has been observed that, as the steam content increases, the average valency of vanadium and the V⁵⁺ content remain practically constant, indicating that this parameter is dependent on the oxygen content. It is observed that the surface area also remains practically constant as the steam content varies in the feed stream.

The crystalline phases of samples 4, 6, 7 and 8 all consist mainly of VPP with traces of ω—VOPO₄, δ-VOPO₄ and VOPO₄·2H₂O. However, catalysts 6 and 7, for the same crystalline phases, are characterized by a clear difference in terms of structural disorder of the (200) lattice plane, which is known to be one of the planes most heavily involved in the oxidation reaction of n-butane, since a lower intensity of XRD reflection was observed than that of catalysts 4 and 8. This greater structural disorder is a symptom of exposure of a higher number of active sites on that lattice plane.

Effect of the transformation temperature of the precursor—Catalyst 9 was activated in an atmosphere composed of air: steam in a 30:70 ratio like the atmosphere used for the reference sample (catalyst 4), modifying the temperature ramp of step 2 with the introduction of a step of holding the temperature at 350° C. for 1 hour. Specifically, to activate catalyst 9, in step 2 heating was carried out from 180° C. to 350° C. at a heating rate of 2° C./min and the temperature was held at 350° C. for 1 hour, followed by heating from 350° C. to 425° C. at a heating rate of 2° C./min and the temperature was held at 425° C. for 2 hours, with a feed stream composed of air: steam in a ratio of 30:70.

In terms of surface area and average valency of vanadium, no significant differences were observed in the comparison between catalyst 9 and the reference sample. With regard to crystalline phases, both catalysts have a main phase of VPP with the presence of ω—VOPO₄, however in sample 9 δ-VOPO₄, a phase known for being advantageously selective for the formation of maleic anhydride, is also present.

Catalysts 10 to 14 were activated starting from precursor B, for the purpose of assessing the reproducibility of the preceding results observed in catalysts 1-9 obtained by activation of the precursor A. The chemical-physical characterization carried out on catalysts 10-14 effectively confirmed the observations made above for catalysts 1-9.

TABLE 3

| | Characterization of fresh catalyst | | | |
|---|---|---|---|---|
| | | BET | | |
| Cat. | Vox | [m²/g] | XRD | RAMAN |
| 1 | 4.52 | 9 | Mainly amorphous, | Main phase |
| | | | traces of VPP, | ω-VOPO₄, traces of |
| | | | VOPO₄•2H₂O and | αI-VOPO₄ |
| | | | ω-VOPO₄ | |
| 2 | 4.53 | 12 | Crystalline, main | Main phase |
| | | | phase ω-VOPO₄, | ω-VOPO₄, traces of |
| | | | traces of | αI-VOPO₄ and |
| | | | VOPO₄•2H₂O and | VOPO₄•2H₂O |
| | | | VPP | |
| 3 | 4.41 | 11 | Crystalline, main | Main phase |
| | | | phase ω-VOPO₄ and | VOPO₄•2H₂O, traces |
| | | | VPP, traces of | of VPP |
| | | | VOPO₄•2H₂O | |
| 4 | 4.21 | 13 | Crystalline, main | Main phase VPP, |
| | | | phase VPP and | traces of |
| | | | ω-VOPO₄ | VOPO₄•2H₂O and |
| | | | | ω-VOPO₄ |
| 5 | 4.47 | 10 | Low crystallinity, | VPP, αI-VOPO₄, |
| | | | ω-VOPO₄ and VPP | ω-VOPO₄ and |
| | | | | VOPO₄•2H₂O |
| 6 | 4.25 | 14 | Low crystallinity, | VOPO₄•2H₂O, VPP |
| | | | main phase | and δ-VOPO₄ |
| | | | ω-VOPO₄ and VPP | |
| 7* | 4.19 | 11 | Crystalline, main | VPP and |
| | | | phase VPP and | VOPO₄•2H₂O |
| | | | ω-VOPO₄ | |
| 8 | 4.23 | 12 | Crystalline, main | VPP, ω-VOPO₄ and |
| | | | phase VPP and | VOPO₄•2H₂O |
| | | | ω-VOPO₄ | |
| 9* | 4.25 | 16 | Crystalline, main | Main phase VPP, |
| | | | phase VPP, | ω-VOPO₄ and |
| | | | ω-VOPO₄ and | δ-VOPO₄ |
| | | | δ-VOPO₄ | |
| 10 | 4.58 | 13 | Low crystallinity, | ω-VOPO₄, traces of |
| | | | main phase | αI-VOPO₄ |
| | | | VOPO₄•2H₂O, traces | |
| | | | of ω-VOPO₄ | |
| 11 | 4.60 | 9 | Crystalline, main | VOPO₄•2H₂O and |
| | | | phase VOPO₄•2H₂O, | ω-VOPO₄ |
| | | | traces of VPP | |
| 12 | 4.51 | 7 | Crystalline, main | Main phase VPP |
| | | | phase VPP, | and VOPO₄•2H₂O |
| | | | VOPO₄•2H₂O and | |
| | | | ω-VOPO₄ | |
| 13 | 4.27 | 17 | Crystalline, main | Main phase VPP, |
| | | | phase VPP and | traces of |
| | | | ω-VOPO₄ | VOPO₄•2H₂O |
| 14* | 4.33 | 19 | Crystalline, main | Main phase VPP |
| | | | phase VPP and | and ω-VOPO₄, |
| | | | ω-VOPO₄ | traces of αI-VOPO₄ |

In Table 3, the catalysts according to the invention are marked with “*”, and the remaining catalysts are comparitives.

### 4) Setup of the Catalytic Tests

The study of the catalytic performance of the catalysts was conducted on a laboratory scale, using a micro-reactor with ID of 1.4 cm inserted into an electric resistance oven under the following reference operating conditions: atmospheric pressure, reaction temperature=400° C., inlet n-butane concentration 1.70 mol %, and GHSV of 2400 h⁻¹.

For each catalytic test, a sample of catalyst of 2.0 g was loaded in the micro-reactor, corresponding to a height of the catalytic bed of 0.64 cm. The thermocouple was placed inside the catalytic bed, at the center (depth≈0.32 cm), and was used to regulate the reaction temperature. Once the micro-reactor was loaded, the catalyst was equilibrated for approximately 50 hours at 400° C. under the same conditions of n-butane and air used during the reaction phase.

The composition of the reaction products in the gaseous phase was analyzed by means of gas chromatography, and the results obtained (shown in Table 4) refer to the reactivity observed with a reaction temperature equal to 400° C.

### 5) Results of the Micro-Reactor Tests

In all the reactivity tests, the reaction temperature was kept constant and equal to 400° C., thus making it possible to carry out a comparison of the results in terms both of conversion of n-butane (n-C4), and of selectivity to the main reaction products, i.e. maleic anhydride (MA) and COₓ. The results of the catalytic tests are reported in Table 4, while Table 5 reports the results of the chemical-physical characterization (using XRD and Raman spectroscopy) carried out on the “exhausted” catalysts unloaded from the micro-reactor after the reaction.

TABLE 4

Catalytic performance @400° C.

| | n-C4 conv. | MA sel. | COx sel. | MA yield |
|---|---|---|---|---|
| Cat. | [mol %] | [mol %] | [mol %] | [mol %] |
| 1 | 11.9 | 19.2 | 67.5 | 2.3 |
| 2 | 12.1 | 34.8 | 51.4 | 4.2 |
| 3 | 12.4 | 57.3 | 32.1 | 7.1 |
| 4 | 23.7 | 56.8 | 42.2 | 13.5 |
| 5 | 41.5 | 53.8 | 34.2 | 22.3 |
| 6 | 40.2 | 60.7 | 38.6 | 24.4 |
| 7* | 40.9 | 77.9 | 21.1 | 31.9 |
| 8 | 22.5 | 56.7 | 42.5 | 12.8 |
| 9* | 19.8 | 75.2 | 21.8 | 14.9 |
| 10 | 23.2 | 60.9 | 25.6 | 14.0 |
| 11 | 20.1 | 60.0 | 22.2 | 12.1 |
| 12 | 31.3 | 64.5 | 21.3 | 20.2 |
| 13 | 41.6 | 73.2 | 18.9 | 30.5 |
| 14* | 52.0 | 75.2 | 18.6 | 39.1 |

In Table 4, the catalysts according to the invention are marked with “*”, and the remaining catalysts are for comparison.

Relative air: steam composition—An examination of the performance 5 of catalysts 1 to 3, activated by modifying the relative composition of the air: steam mixture with respect to the 30:70 composition used to activate catalyst 4 (reference sample), shows that by increasing the steam content, or decreasing the amount of air (oxygen), both the activity of the catalyst and its selectivity for MA are increased.

With catalyst 1 there is a 11.9% conversion rate of n-butane and a selectivity for MA of only 19.2%. Catalysts 2 and 3 show a conversion rate of approximately 12%, but with a marked increase in selectivity for MA, which assumes values of respectively 34.8% and 57.3%. Performance is therefore lower than that of the reference sample: catalyst 4, with a conversion rate of 23.7% and a selectivity of 56.8%, is better than catalysts 1-3.

The above catalytic performance levels are in line with the chemical-physical characterization carried out on the fresh catalysts, from which it has been seen that the active catalyst is increasingly richer in the active phase VPP and in traces of ω—VOPO₄ and δ-VOPO₄ when the steam content is increased and the quantity of air is simultaneously decreased. Upon subjecting the catalysts unloaded from the reactor, post-reaction, to a new chemical-physical characterization, it was observed that catalysts 3 and 4, and in particular the latter, contain, in addition to VPP, also δ-VOPO₄, a phase that is selective for the formation of MA.

Effect of the partial pressure of oxygen-Analysis of the performance of catalysts 1, 5 and 6 reveals that decreasing the percentage of oxygen in the feed stream during activation (respectively from 21.0% to 12.6% and 6.3%) results in a marked increase in catalytic activity with a parallel increase in selectivity for MA: the conversion of butane in fact goes from 11.9% for catalyst 1 to 41.5% and 40.2% for catalysts 5 and 6, while the selectivity increases from 19.2% (catalyst 1) to 53.8% and 60.7 (respectively catalyst 5 and 6). In this manner, an increase of the MA yield from 2.3% to 24.4% is obtained.

Also in the case of the effect of the partial pressure of oxygen, the results obtained in the micro-reactor are in line with the chemical-physical characterization, for which it has been seen that, as the percentage of oxygen during activation decreases, the content of active phase VPP and of the δ-VOPO₄ species which is selective for the formation of MA, increases,

As a consequence of these observations, the inventors have concluded that the optimal value of the partial pressure of oxygen coincides with the use of an atmosphere containing 30% air during the activation. It is for this reason that the tests to assess the effect of the partial pressure of steam were carried out while keeping the quantity of air constant at 30%.

Effect of the partial pressure of steam—Comparison of the catalytic performance of catalysts 4, 6, 7 and 8 shows that as the steam content varies (going from catalyst 6, which is activated in the absence of steam, to catalysts 7, 8 and 4, which are activated respectively with 10%, 40% and 70% steam) the best catalytic performance, both in terms of conversion of n-butane and of selectivity for MA, is obtained with catalysts 6 and 7, which are activated with the lower quantity of steam. Catalysts 6 and 7 in fact show a conversion of butane of approximately 40% compared to approximately 23% for catalysts 4 and 8, while in terms of selectivity the value of 77.9% registered with catalyst 7 is the best.

In this case too, the chemical-physical characterization is in line with the catalytic performance. The catalyst with the best performance (catalyst 7) is in fact the one characterized by VPP as the main crystalline phase, with the presence of ω—VOPO₄, and by the highest degree of disorder of the (200) lattice plane.

TABLE 5

| | Characterization of exhausted catalyst | |
|---|---|---|
| Cat. | XRD | RAMAN |
| 1 | Main phase δ-VOPO₄, traces | Main phase δ-VOPO₄, traces |
| | of VPP | of VOPO₄•2H₂O |
| 2 | Main phase δ-VOPO₄, traces | Main phase δ-VOPO₄, traces |
| | of VPP | of VOPO₄•2H₂O |
| 3 | Main phase δ-VOPO₄, traces | Main phase δ-VOPO₄, traces |
| | of VPP | of VOPO₄•2H₂O and VPP |
| 4 | Main phase VPP and | Main phase VPP, δ-VOPO₄ |
| | ω-VOPO₄, traces of δ-VOPO₄ | and αI-VOPO₄ |
| 5 | Main phase VPP | Main phase VPP |
| 6 | Main phase VPP | Main phase VPP |
| 7* | Main phase VPP | Main phase VPP, traces of |
| | | αI-VOPO₄ |
| 8 | Main phase VPP | Main phase VPP, traces of |
| | | αI-VOPO₄ |
| 9* | Main phase VPP | Main phase VPP, traces of |
| | | αI-VOPO₄ |
| 10 | Main phase δ-VOPO₄ | δ-VOPO₄, traces of |
| | | VOPO₄•2H₂O |
| 11 | Main phase δ-VOPO₄, traces | δ-VOPO₄, traces of |
| | of VPP | VOPO₄•2H₂O |
| 12 | Main phase VPP and | Main phase VPP, δ-VOPO₄ |
| | δ-VOPO₄ | and VOPO₄•2H₂O |
| 13 | Main phase VPP, δ-VOPO₄ | Main phase VPP and |
| | and ω-VOPO₄ | δ-VOPO₄ |
| 14* | Main phase VPP and | Main phase VPP, traces of |
| | ω-VOPO₄ | αI-VOPO₄ |

In Table 5, the catalysts according to the invention are marked with “*”, and the remaining catalysts are comparative.

### CONCLUSIONS

Analysis of the data obtained in the micro-reactor and the results of the chemical-physical characterization have led to the conclusion that it is possible to obtain a VPO catalyst with improved catalytic performance when the activation process currently used (Table 2, catalyst 4) to convert the precursor to VPP is modified according to the present invention, i.e. by using in step 2 an atmosphere composed of air: steam: nitrogen in the ratio of 20-40:5-15:45-75, preferably 30:10:60 with no changes to the temperature ramp, or changing the temperature ramp of step 2 by holding at a temperature of 320-380° C., preferably 350° C., for 0.5-3 hours, preferably 1 hour.

In practice it has been found that the process of transformation of the precursor into the active catalyst according to the invention fully achieves the set aim, in that it makes it possible to obtain an active catalyst for the partial oxidation of n-butane to maleic anhydride that—with respect to the current generation of commercial VPO catalysts—is characterized by an improvement in catalytic performance.

Finally, it has also been observed that the present invention fulfills the object of providing a process for producing maleic anhydride with high yield and selectivity.

The disclosures in Italian Patent Application No. 102022000003872 from which this application claims priority are incorporated herein by reference.

1.-10. (canceled)

11. A process for the transformation of a vanadium and phosphorus mixed oxide catalyst precursor into an active catalyst for the synthesis of maleic anhydride,
wherein the precursor comprises vanadyl acid orthophosphate hemihydrate of formula (VO)HPO₄·0.5H₂O as main component and the active catalyst comprises vanadyl pyrophosphate of formula (VO)₂P₂O₇ as main component, and
wherein the process comprises the steps of:
a) initial heating of the precursor to an initial temperature of 170-190° C. and holding at the initial temperature for 1-10 minutes, in an atmosphere composed of 100% by volume of air;
b′) optional heating of the precursor from the initial temperature to a calcination temperature TC1 of 320-380° C. and holding at the temperature TC1 for 0.5-3 hours, in an atmosphere selected from an atmosphere composed of 10%-50% by volume of air and 50%-90% by volume of steam, and an atmosphere composed of 20%-40% by volume of air, 5%-15% by volume of steam and 45%-75% by volume of an inert gas;
b) further heating of the precursor from the initial temperature or, if step b′) is carried out, from the calcination temperature TC1 to a calcination temperature TC2 of 390-460° C. and holding at the temperature TC2 for 0.5-3 hours, in an atmosphere which
1. has the same composition as the atmosphere of step b′), if step b′) is carried out,
2. is composed of 20%-40% by volume of air, 5%-15% by volume of steam and 45%-75% by volume of an inert gas, if step b′) is not carried out;
c) heating of the precursor from the calcination temperature TC2 to a calcination temperature TC3 of 470-550° C. and holding at the calcination temperature TC3 for 0.5-3 hours, in an atmosphere composed of 100% by volume of an inert gas; and
d) cooling the active catalyst obtained in step c).

12. The process according to claim 11, wherein step b′) is carried out, the precursor is heated to the calcination temperature TC1 of 320-380° C., preferably 350° C., and the calcination temperature TC1 is held for 0.5-3 hours, preferably 1-2 hours, more preferably 1 hour, in an atmosphere composed of 10%-50% by volume of air and 50%-90% by volume of steam, preferably composed of 30% by volume of air and 70% by volume of steam.

13. The process according to claim 11, wherein step b′) is carried out, the precursor is heated to the calcination temperature TC1 of 320-380° C., preferably 350° C., and the calcination temperature TC1 is held for 0.5-3 hours, preferably 1-2 hours, more preferably 1 hour, in an atmosphere composed of 20%-40% by volume of air, 5%-15% by volume of steam and 45%-75% by volume of an inert gas, preferably composed of 30% by volume of air, 10% by volume of steam and 60% by volume of an inert gas.

14. The process according to claim 11, wherein step b′) is not carried out and in step b) the precursor is heated to the calcination temperature TC2 of 390-460° C., preferably 425° C., and the calcination temperature TC2 is held for 0.5-3 hours, preferably 1-2 hours, more preferably 2 hours, in an atmosphere composed of 20%-40% by volume of air, 5%-15% by volume of steam and 45%-75% by volume of an inert gas, preferably composed of 30% by volume of air, 10% by volume of steam and 60% by volume of an inert gas.

15. The process according to claim 11, wherein in step a) the precursor is heated to the initial temperature of 180° C. and held at the initial temperature for 2 minutes.

16. The process according to claim 11, wherein in step c) the precursor is heated to the calcination temperature TC3 of 500° C. and held at the calcination temperature TC3 for 2 hours.

17. The process according to claim 11, wherein in each of steps a), b′), b) and c), independently of each other, heating is carried out at a heating rate from 0.1° C./minute to 10° C./minute, preferably 2° C./minute.

18. The process according to claim 11, wherein the precursor is precalcined prior to step a), preferably at a precalcination temperature of 200-300° C. in an atmosphere of air.

19. An active catalyst for the synthesis of maleic anhydride, obtainable using the process according to claim 11.

20. A process for the production of maleic anhydride by partial oxidation of n-butane in an oxygen-containing gas mixture in the presence of the active catalyst according to claim 19.
